(19) United States
(12) Patent Application Publication
(10) Pub. No.: US 2025/0308694 A1
(43) Pub. Date: Oct. 2, 2025

(54) METHOD AND TOOL FOR ASSISTING CLINICIANS IN MAKING REAL-TIME DECISIONS FOR NEONATAL SHOCK SYNDROMES

(71) Applicant: Cloudphysician Healthcare Pvt Ltd, Bangalore (IN)

(72) Inventors: Carl Britto, Bengaluru (IN); Dhruv Sud, Bengaluru (IN)

(21) Appl. No.: 19/091,964

(22) Filed: Mar. 27, 2025

(30) Foreign Application Priority Data
Mar. 27, 2024 (IN) 202441024609

(51) Int. Cl. G16H 50/20; G16H 10/20; G16H 10/40; G16H 10/60

(52) U.S. Cl. CPC G16H 50/20; G16H 10/20; G16H 10/40; G16H 10/60

(57) ABSTRACT

This disclosure relates to method and tool assisting clinicians in making real time decisions for resuscitation modalities in neonatal shock syndromes. The method includes receiving medical data corresponding to a neonate diagnosed with shock. The method further includes extracting one or more features from the medical data. The one or more features are indicative of perfusion status, possible etiology, type, and severity of shock, in the neonate. Further, the method includes selecting at least one machine learning model (ML) from a plurality of ML models based on the one or more features. Further, the method may include predicting via at least one ML model, an optimal treatment modality for the neonate. The method further includes assisting a clinician to provide the optimal treatment modality to the neonate based on predicting.

### TECHNICAL FIELD

This disclosure generally relates to a field of pediatric health care. More particularly the present disclosure relates to method and tool for assisting clinicians in making real time decisions for resuscitation modalities in neonatal (premature infants and those under the age of 30 days) shock syndromes.

### BACKGROUND

Neonatal shock syndromes represent a critical and life-threatening condition that affects preterm infants and term newborns, often necessitating rapid and precise medical interventions to ensure the best possible outcome. Neonates diagnosed with shock face significant challenges related to cardiovascular function and fluid balance, which may lead to inadequate perfusion of vital organs. While the occurrence of neonatal shock is not uncommon in neonatal intensive care units, the approach to its diagnosis and management remains a complex and multifaceted issue.

Traditionally, the management of neonatal shock has lacked standardization, with clinical protocols varying widely across healthcare facilities. Clinicians, typically neonatologists, face the formidable task of evaluating a neonate's condition, determining the underlying etiology of shock, and deciding on the most appropriate resuscitation modalities. These modalities often involve a delicate balance between administering fluid boluses and initiating pressor support. This balance is crucial since most neonates with shock also have tenuous respiratory states and excess fluid administration can result in rapid deterioration.

The absence of clear, data-driven guidelines and evidence-based approaches has added to the complexity of decision-making in neonatal shock cases. Therefore, there is a need to develop a method and a tool that is capable of assisting clinicians in selecting optimal first-line fluid and pressor modalities for resuscitation in neonatal shock syndromes. Through the utilization of appropriate machine learning (ML) techniques (for example, decision trees or random forests, support vector machines (SVMs), recurrent neural networks (RNNs) or long short-term memory (LSTM), transformers, and Bayesian methods) and analysis of neonate medical data, the proposed method and tool provide a data-driven approach to decision-making.

### SUMMARY

In one embodiment, a method for assisting clinicians in making real time decisions for resuscitation modalities in neonatal shock syndromes is disclosed. The method may include receiving medical data corresponding to a neonate diagnosed with shock. The method further may include extracting one or more features from the medical data. The one or more features are indicative of perfusion status, possible etiology, type, and severity of shock, in the neonate. Further, the method may include selecting at least one machine learning model (ML) from a plurality of ML models based on the one or more features. The plurality of ML models may include decision trees or random forests, support vector machines (SVMs), recurrent neural networks (RNNs) or long short-term memory (LSTM), transformers, and Bayesian methods. Further, the method may include predicting via at least one ML model, an optimal treatment modality for the neonate. The optimal treatment modality may be one of fluid boluses or pressor support. The method further includes assisting a clinician to provide the optimal treatment modality to the neonate based on predicting. Assisting may specify a number and type of fluid boluses for the neonate, or a type and dose of the pressor support for the neonate. The assisting may be based on the etiology and severity of shock in the neonate.

In another embodiment, a tool for assisting clinicians in making real time decisions for resuscitation modalities in neonatal shock syndromes is disclosed. The system may include a processor and a computer-readable medium communicatively coupled to the processor. The computer-readable medium may store processor-executable instructions, which, on execution, may cause the processor to receive medical data corresponding to a neonate diagnosed with shock. The processor-executable instructions, on execution, may further cause the processor to extract one or more features from the medical data. The one or more features are indicative of perfusion status, etiology, type, and severity of shock,, in the neonate. The processor-executable instructions, on execution, may further cause the processor to select at least one machine learning model (ML) from a plurality of ML models based on the one or more features. The plurality of ML models may include decision trees or random forests, support vector machines (SVMs), recurrent neural networks (RNNs) or long short-term memory (LSTM), transformers, and Bayesian methods. The processor-executable instructions, on execution, may further cause the processor to predict via at least one ML model, an optimal treatment modality for the neonate. The optimal treatment modality may be one of fluid boluses or pressor support. The processor-executable instructions, on execution, may further cause the processor to assist a clinician to provide the optimal treatment modality to the neonate based on predicting. Assisting may specify a number and type of the fluid boluses for the neonate, or a type and dose of the pressor support for the neonate. The assisting may be based on the etiology and severity of shock in the neonate.

In yet another embodiment, a non-transitory computer-readable medium storing computer-executable instruction for assisting clinicians in making real time decisions for resuscitation modalities in neonatal shock syndromes is disclosed. The stored instructions, when executed by a processor, may cause the processor to perform various operations including receiving medical data corresponding to a neonate diagnosed with shock. The operations may further include extracting one or more features from the medical data. The one or more features are indicative of perfusion status, etiology, type, and severity of shock,, in the neonate. Further, the operations may include selecting at least one machine learning model (ML) from a plurality of ML models based on the one or more features. The plurality of ML models may include decision trees or random forests, support vector machines (SVMs), recurrent neural networks (RNNs) or long short-term memory (LSTM), transformers, and Bayesian methods. Further, the operations may include predicting at least one ML model, an optimal treatment modality for the neonate. The optimal treatment modality may be one of fluid boluses or pressor support. The operations may further include assisting a clinician to provide the optimal treatment modality to the neonate based on predicting. Assisting may specify a number and type of the fluid boluses for the neonate, or a type and dose of the pressor support for the neonate. The assisting may be based on the etiology and severity of shock in the neonate.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for assisting clinicians in making real time decisions for resuscitation modalities in neonatal shock syndromes, in accordance with some embodiments.

FIG. 2 is a functional block diagram of various modules within a memory of a clinical decision support tool, in accordance with some embodiments.

FIG. 3 is a flow diagram of a method for assisting clinicians in making real time decisions for resuscitation modalities in neonatal shock syndromes, in accordance with some embodiments.

FIG. 4 is a flow diagram of a method for training an ML model, in accordance with some embodiments.

FIG. 5 is a flow diagram of a method for optimizing an ML model, in accordance with some embodiments.

FIG. 6 is a block diagram of an exemplary computer system for implementing embodiments consistent with the present disclosure, in accordance with some embodiments.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

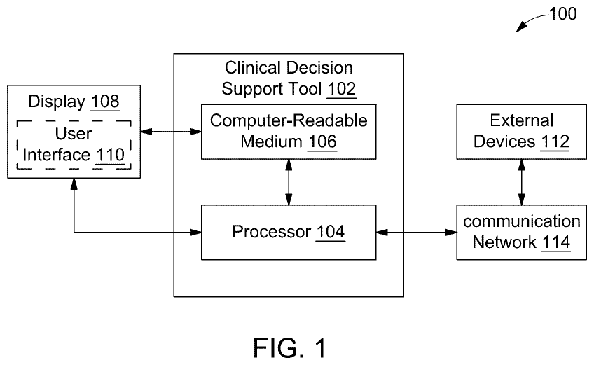

Referring now to FIG. 1, an exemplary system 100 for assisting clinicians in making real time decisions for resuscitation modalities in neonatal shock syndromes is illustrated, in accordance with some embodiments. The system 100 may include a clinical decision support tool 102 that may be responsible to assist clinicians (for example, neonatologist) in making real time decisions for resuscitation modalities in neonatal shock syndromes. The clinical decision support tool 102 may be, for example, a server, a desktop, a laptop, a notebook, a netbook, a tablet, a smartphone, a mobile phone, or any other computing device.

The present disclosure may revolutionize the clinical management of neonatal shock syndromes, offering clinicians a powerful tool (for example, the clinical decision support tool 102) to make timely and informed decisions. By providing clear and personalized guidance based on medical data analysis, it has a potential to improve outcomes for neonates facing this critical medical condition and to standardize the approach to resuscitation in neonatal intensive care units.

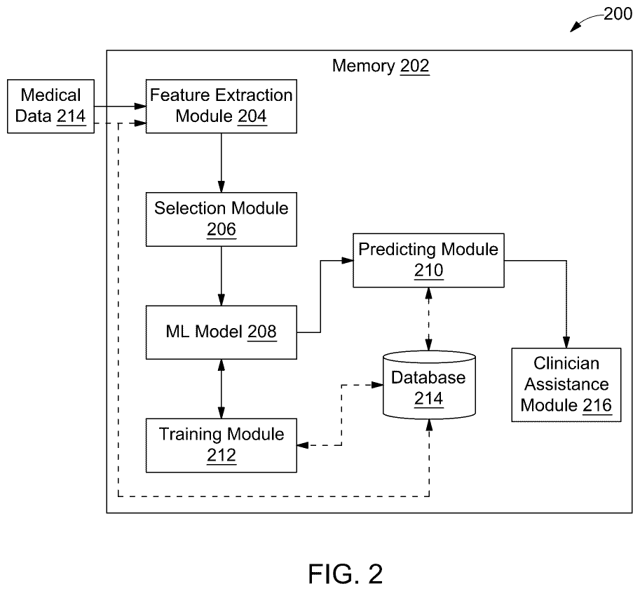

As will be described in greater detail in conjunction with FIGS. 2-5, in order to assist clinicians in making real time decisions for resuscitation modalities in neonatal shock syndromes, the clinical decision support tool 102 may receive medical data corresponding to a neonate diagnosed with shock. The medical data may include, but may not be limited to, medical history of the neonate, vital signs of the neonate, laboratory results of the neonate, and other related clinical and demographic information of the neonate. The medical data may be received from one or more electronic health records, bedside monitors, laboratory tests, imaging devices, wearable sensors, and the like. The clinical decision support tool 102 may further extract one or more features from the medical data. The one or more features may be indicative of perfusion status, possible etiology, type, and severity of shock, in the neonate. The clinical decision support tool 102 may further select at least one machine learning model (ML) from a plurality of ML models based on the one or more features. The plurality of ML models may include decision trees or random forests, support vector machines (SVMs), recurrent neural networks (RNNs) or long short-term memory (LSTM), transformers, and Bayesian methods. The clinical decision support tool 102 may further predict, via at least one ML model, an optimal treatment modality for the neonate. The optimal treatment modality may be one of fluid boluses or pressor support. The clinical decision support tool 102 may further assist a clinician to provide the optimal treatment modality to the neonate based on the predicting. It may be noted that assisting may specify a number and type of fluid boluses for the neonate, or a type and dose of the pressor support for the neonate. Assisting may be based on the etiology and severity of shock in the neonate.

In some embodiments, the clinical decision support tool 102 may include one or more processors 104 and a computer-readable medium 106 (for example, a memory). The computer-readable storage medium 106 may store instructions that, when executed by the one or more processors 104, cause the one or more processors 104 to assist clinicians in making real time decisions for resuscitation modalities in neonatal shock syndromes, in accordance with aspects of the present disclosure. The computer-readable storage medium 106 may also store various data (for example, medical data (such as medical history of the neonate, vital signs of the neonate, laboratory results of the neonate, and other related clinical and demographic information of the neonate), training data, real-time medical data (such as live data of the neonate) and the like) that may be captured, processed, and/or required by the system 100.

The system 100 may further include a display 108. The system 100 may interact with a user via a user interface 110 accessible via the display 108. The system 100 may also include one or more external devices 112. In some embodiments, the clinical decision support tool 102 may interact with the one or more external devices 112 over a communication network 114 for sending or receiving various data. The external devices 112 may include, but may not be limited to, a remote server, a digital device, or any other devices commonly used in neonatal intensive care units. The clinical decision support tool 102 may interact with these external devices to access additional patient data or to send information related to their decisions.

Referring now to FIG. 2, a functional block diagram 200 of various modules within a memory 202 of the clinical decision support tool 102 is illustrated, in accordance with some embodiments. In particular, the clinical decision support tool 102 may include, within the memory 202, a feature extraction module 204, a selection module 206, an ML model 208, a prediction module 210, a training module 212, a database 214, and a clinician assistance module 216. The memory 202 may receive a medical data 218. In some embodiments, the memory 202 may be analogous to the computer-readable medium 106 implemented by the system 100.

The feature extraction module 204 may receive the medical data 218 corresponding to a neonate diagnosed with shock. The medical data 218 may include medical history of the neonate, vital signs of the neonate, laboratory results of the neonate, and other related clinical and demographic information of the neonate. It should be noted that the medical data may be received from one or more of electronic health records, bedside monitors, laboratory tests, imaging devices, or wearable sensors.

The selection module 206 may select at least one machine learning model (ML) 208 from a plurality of ML models based on the one or more features. The plurality of ML models may include decision trees or random forests, support vector machines (SVMs), recurrent neural networks (RNNs) or long short-term memory (LSTM), transformers, and Bayesian methods. In some embodiments, a combination of ML models may be selected. For example, the decision trees or random forests to prevent over-fitting. Support vector machines for patient classification for treatment modality depending on the etiology. Gradient boosting for treatment recommendations. Neural networks (RNN/LTSM) for complex relationships between features. We also use Bayesian approaches to probabilistically validate with past patients data.

Once the ML model is selected, the prediction module 210 may employ the selected ML model to predict an optimal treatment modality for the neonate. It should be noted that the optimal treatment modality may be one of fluid boluses or pressor support.

In some embodiments, the selected ML model 208 may be trained on the received medical data and extracted features. After training the ML model 208, it would need to be validated using new data to evaluate its accuracy in predicting whether a neonate should receive fluid boluses or pressor support.

In some embodiments, the ML model 208 may be optimized to improve its performance by adjusting hyperparameters, selecting different features, or trying different ML algorithms/combinations. Once the model has been optimized, it may be deployed in clinical settings to assist clinicians in making treatment decisions for neonates diagnosed with shock.

Based on predicting, the clinician assistance module 216 may assist the clinician to provide the optimal treatment modality to the neonate. The assisting may specify a number and type of fluid boluses for the neonate, or a type and dose of the pressor support for the neonate, and the assisting is based on the etiology and severity of shock in the neonate. The database 214 may store medical data (such as medical history of the neonate, vital signs of the neonate, laboratory results of the neonate, and other related clinical and demographic information of the neonate), training data, real-time medical data received from one or more of electronic health records, bedside monitors, laboratory tests, imaging devices, or wearable sensors.

It should be noted that all such aforementioned modules 204-216 may be represented as a single module or a combination of different modules. Further, as will be appreciated by those skilled in the art, each of the modules 204-216 may reside, in whole or in parts, on one device or multiple devices in communication with each other. In some embodiments, each of the modules 204-216 may be implemented as a dedicated hardware circuit comprising of custom application-specific integrated circuit (ASIC) or gate arrays, off-the-shelf semiconductors such as logic chips, transistors, or other discrete components. Each of the modules 204-216 may also be implemented in a programmable hardware device such as a field programmable gate array (FPGA), programmable array logic, programmable logic device, and so forth. Alternatively, each of the modules 204-216 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together, but may include disparate instructions stored in different locations which, when joined logically together, include the module, and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for assisting clinicians in making real time decisions for resuscitation modalities in neonatal shock syndromes. For example, the exemplary system 100 and the associated clinical decision support tool 102 may assist clinicians in making real time decisions through an ML model by the processes discussed herein. In particular, as will be appreciated by those of ordinary skill in the art, control logic and/or automated routines for performing the techniques and steps described herein may be implemented by the system 100 and the associated clinical decision support tool 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some, or all of the processes described herein may be included in the one or more processors on the system 100.

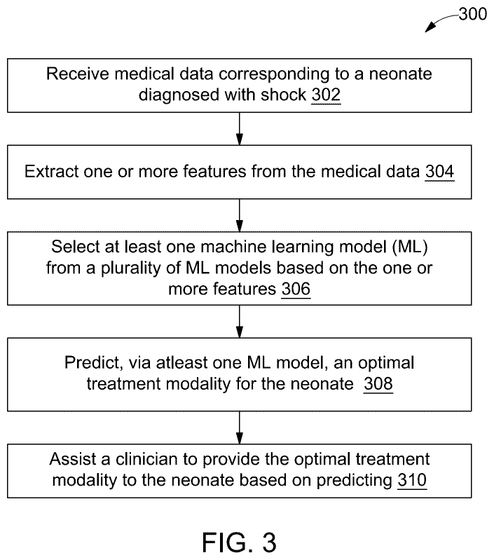

Referring to FIG. 3, a flow diagram of a method 300 for assisting clinicians in making real time decisions for resuscitation modalities in neonatal shock syndromes is illustrated, in accordance with some embodiments. The method 300 may be implemented by the clinical decision support tool 102 of the system 100. The method 300 includes receiving medical data corresponding to a neonate diagnosed with shock, at step 302. It may be noted that the medical data may be received from one or more electronic health records, bedside monitors, laboratory tests, imaging devices, or wearable sensors. The medical data may include medical history of the neonate, vital signs of the neonate, laboratory results of the neonate, and other related clinical and demographic information of the neonate.

Further, method 300 includes extracting one or more features from the medical data, at step 304. The one or more features may be indicative of perfusion status, possible etiology, type, and severity of shock, in the neonate.

Further, method 300 includes selecting at least one machine learning model (ML) from a plurality of ML models based on the one or more features, at step 306. The plurality of ML models may include decision trees or random forests, support vector machines (SVMs), gradient boosting, recurrent neural networks (RNNs) or long short-term memory (LSTM), transformers, and Bayesian methods. By way of an example, the selection module 206 may select an appropriate ML model or a combination of ML models from the plurality of ML models, for example, decision trees or random forests to prevent over-fitting, SVMs for patient classification for treatment modality depending on the etiology, gradient boosting for treatment recommendations, RNNs or LSTM for complex relationships between features, and Bayesian methods to probabilistically validate with past patients data.

Further, method 300 includes predicting, via at least one ML model, an optimal treatment modality for the neonate, at step 308. The optimal treatment modality may be one of fluid boluses or pressor support.

Further, step 310 includes assisting a clinician to provide the optimal treatment modality to the neonate based on the prediction. It should be noted that assisting specifies a number and type of fluid boluses for the neonate, or a type and dose of the pressor support for the neonate. The assisting may be based on the etiology and severity of shock in the neonate.

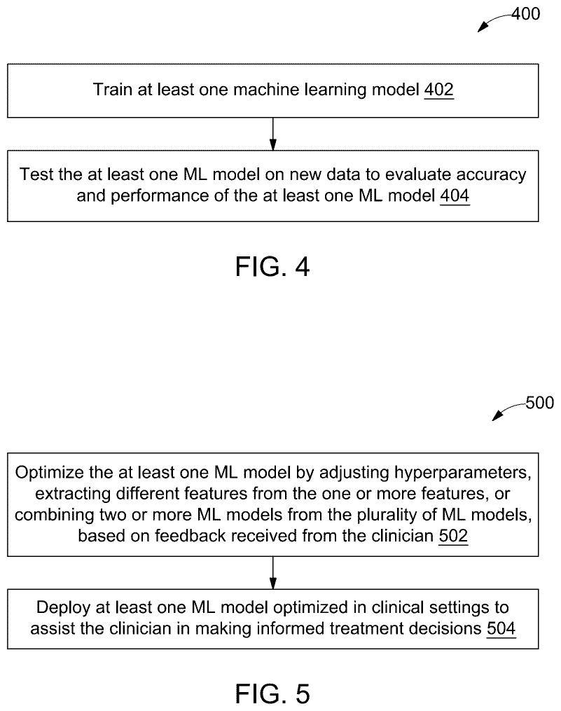

Referring to FIG. 4, a flow diagram of a method 400 for training an ML model (for example, the ML model 208) is illustrated, in accordance with some embodiments. The method 400 may be implemented by the clinical decision support tool 102 of the system 100. Method 400 includes training at least one ML model, at step 402. The at least one ML model may be trained on a dataset of the medical data and the one or more features extracted. This training phase equips the ML model with the ability to make predictions based on the patterns and information contained within the dataset.

Further, method 400 includes testing the at least one ML model on new data to evaluate accuracy and performance of the at least one ML model, at step 404. In other words, after the ML model has been trained in step 402, the next step is to assess their performance using new and previously unseen data. The new data may be data that has been collected or generated after the ML models completed their training. It represents information that the ML model has never been exposed to before. The new data reflects real-world cases or scenarios that have arisen since the model were last trained. It is essential for evaluating how well the ML model generalize to new situations.

Additionally, the previously unseen data may be the data that existed at the time of training but was intentionally kept separate from the training dataset. ML models are typically trained on a specific dataset to learn patterns and relationships within that data. Previously unseen data may be withheld from the ML models during training to serve as an independent evaluation dataset. It ensures that the ML models are assessed on cases they have not seen before, helping to estimate their ability to make accurate predictions in real-world situations.

This evaluation may be essential to ensure that the model may generalize well and make accurate predictions when applied to real-world scenarios. In particular, the evaluation may help to validate the reliability and robustness of the ML model. It verifies whether the model's performance during training was indicative of its actual capabilities and whether it may consistently make accurate predictions across different datasets and scenarios.

For neonatal shock syndromes, clinical validation is especially important. It involves comparing ML model recommendations with the actual treatment decisions made by clinicians in a clinical setting. The success of the proposed tool is ultimately measured by its ability to assist clinicians effectively in making real-time treatment decisions for neonatal shock cases.

Referring now to FIG. 5, a flow diagram of a method 500 for optimizing an ML model (for example, the ML model 208) is illustrated, in accordance with some embodiments. The method 500 may be implemented by the clinical decision support tool 102 of the system 100. Method 500 includes optimizing the at least one ML model, at step 502. The optimization may be done by adjusting hyperparameters, extracting different features from the one or more features, or combining two or more ML models from the plurality of ML models, based on feedback received from the clinician.

In a more elaborative way, one aspect of optimization involves fine-tuning the model's hyperparameters. Hyperparameters are configurable settings that influence the model's learning process but are not learned from the data. They include parameters like learning rates, regularization strengths, and architectural choices. By adjusting these hyperparameters based on feedback, the ML model may be optimized for improved accuracy and reliability.

Further, feature engineering and selection are key for optimizing model performance. This may involve revisiting the set of features used as input to the ML model. The features may be added, removed, or transformed to enhance their relevance and effectiveness in capturing critical patterns in the data. Feature optimization aims to ensure that the ML model may make more informed predictions based on the available information.

In certain scenarios, optimizing the ML model may involve combining the predictions of multiple ML models from the plurality of ML models. Model combination techniques, such as ensemble methods, are employed to integrate the strengths of different models. This approach may enhance prediction accuracy and robustness.

Additionally, clinician feedback is a keystone of the optimization process. Feedback received from healthcare professionals is invaluable as it provides real-world understandings to the effectiveness of the ML model in clinical settings. The clinicians may offer feedback on the relevance of recommendations, the usability of the clinical decision support tool, and the overall quality of predictions. This feedback loop ensures that the ML model continuously evolve to meet the evolving needs of healthcare providers.

Further, method 500 includes deploying at least one ML model optimized in clinical settings to assist the clinician in making informed treatment decisions, at step 504. Once deployed, the ML model are ready to provide real-time decision support to clinicians. The ML model may be equipped to analyze incoming data related to neonatal shock cases and offer timely recommendations. These recommendations may include treatment modalities, such as optimal fluid boluses or pressor support, along with their corresponding dosages.

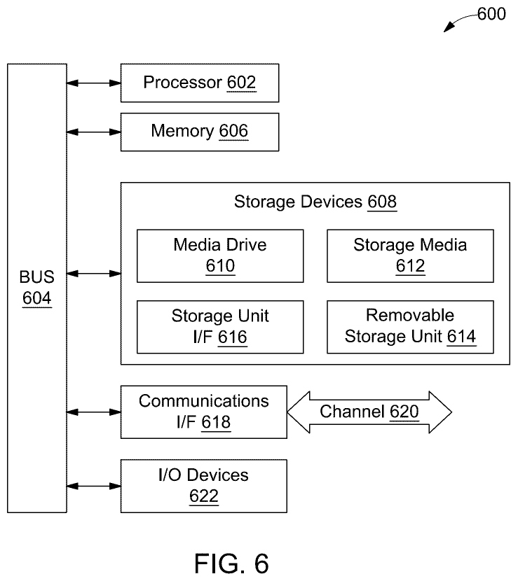

The disclosed methods and systems may be implemented on a conventional or a general-purpose computer system, such as a personal computer (PC) or server computer. Referring now to FIG. 6, an exemplary computing system 600 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 600 may represent, for example, a user device such as a desktop, a laptop, a mobile phone, personal entertainment device, DVR, and so on, or any other type of special or general-purpose computing device as may be desirable or appropriate for a given application or environment. The computing system 600 may include one or more processors, such as a processor 602 that may be implemented using a general or special purpose processing engine such as, for example, a microprocessor, microcontroller, or other control logic. In this example, the processor 602 is connected to a bus 604 or other communication medium. In some embodiments, the processor 602 may be an Artificial Intelligence (AI) processor, which may be implemented as a Tensor Processing Unit (TPU), or a graphical processor unit, or a custom programmable solution Field-Programmable Gate Array (FPGA).

The computing system 600 may also include a memory 606 (main memory), for example, Random Access Memory (RAM) or other dynamic memory, for storing information and instructions to be executed by the processor 602. The memory 606 also may be used for storing temporary variables or other intermediate information during execution of instructions to be executed by the processor 602. The computing system 600 may likewise include a read only memory (“ROM”) or other static storage device coupled to bus 604 for storing static information and instructions for the processor 602.

The computing system 600 may also include a storage device 608, which may include, for example, a media drives 610, a cloud based storage, a network storage, and a removable storage interface. The media drive 610 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 606 may include, for example, a hard disk, magnetic tape, flash drive, or other fixed or removable medium that is read by and written to by the media drive 620. As these examples illustrate, the storage media 612 may include a computer-readable storage medium having stored there in particular computer software or data.

In alternative embodiments, the storage devices 608 may include other similar instrumentalities for allowing computer programs or other instructions or data to be loaded into the computing system 600. Such instrumentalities may include, for example, a removable storage unit 614 and a storage unit interface 616, such as a program cartridge and cartridge interface, a removable memory (for example, a flash memory or other removable memory module) and memory slot, and other removable storage units and interfaces that allow software and data to be transferred from the removable storage unit 614 to the computing system 600.

The computing system 600 may also include a communications interface 418. The communications interface 618 may be used to allow software and data to be transferred between the computing system 600 and external devices. Examples of the communications interface 618 may include a network interface (such as an Ethernet or other NIC card), a communications port (such as for example, a USB port, a micro USB port), Near field Communication (NFC), etc. Software and data transferred via the communications interface 618 are in the form of signals which may be electronic, electromagnetic, optical, or other signals capable of being received by the communications interface 618. These signals are provided to the communications interface 618 via a channel 620. The channel 620 may carry signals and may be implemented using a wireless medium, wire or cable, fiber optics, or other communications medium. Some examples of the channel 620 may include a phone line, a cellular phone link, an RF link, a Bluetooth link, a network interface, a local or wide area network, and other communications channels.

The computing system 600 may further include Input/Output (I/O) devices 622. Examples may include, but are not limited to a display, keypad, microphone, audio speakers, vibrating motor, LED lights, etc. The I/O devices 622 may receive input from a user and also display an output of the computation performed by the processor 602. In this document, the terms “computer program product” and “computer-readable medium” may be used generally to refer to media such as, for example, the memory 606, the storage devices 608, the removable storage unit 614, or signal(s) on the channel 620. These and other forms of computer-readable media may be involved in providing one or more sequences of one or more instructions to the processor 602 for execution. Such instructions, generally referred to as “computer program code” (which may be grouped in the form of computer programs or other groupings), when executed, enable the computing system 600 to perform features or functions of embodiments of the present invention.

In an embodiment where the elements are implemented using software, the software may be stored in a computer-readable medium and loaded into the computing system 600 using, for example, the removable storage unit 614, the media drive 610 or the communications interface 618. The control logic (in this example, software instructions or computer program code), when executed by the processor 602, causes the processor 602 to perform the functions of the invention as described herein.

Various embodiments provide method and tool for assisting clinicians in making real time decisions for resuscitation modalities in neonatal shock syndromes. The disclosed method and tool may collect and process a large amount of medical data to evaluate the perfusion status, possible etiology, type, and severity of shock, in the neonate. By employing an appropriate ML model from a range of ML models, including decision trees, support vector machines, gradient boosting, recurrent neural networks, transformers, and Bayesian methods, the tool predicts most suitable treatment modality. This prediction encompasses the determination of whether to prioritize fluid boluses and specifies the required number or recommends the direct use of pressors, even specifying their starting doses.

As will be appreciated by those skilled in the art, the clinical decision support tool described in the various embodiments discussed above are not routine, or conventional, or well understood in the art. The tool discussed above leverages various machine learning approaches to analyze an extensive array of patient data. This enables clinicians to make more precise and data-driven treatment decisions, potentially leading to improved outcomes for neonates with shock. By providing recommendations grounded in data analysis, the tool empowers clinicians to make well-informed choices in a complex medical context.

Further, the disclosed clinical decision support tool offers standardized treatment recommendations based on data-driven perceptions. By promoting consistency in care practices across different healthcare facilities, it helps to reduce the risk of variations in clinical approaches and enhances the overall quality of patient care.

Timeliness is crucial in medical emergencies, and the disclosed tool operates in real-time. Neonatologist may access immediate guidance and recommendations, which may particularly be valuable in high-pressure situations where rapid decisions are vital. This real-time support may significantly enhance the ability of healthcare professionals to respond effectively to neonatal shock cases. Further, the neonatal shock cases may vary widely in terms of their underlying causes and severity. The tool may consider individual patient data, including perfusion status, etiology, and shock severity, to tailor treatment recommendations. This personalized approach may lead to more effective and patient-centered care, addressing the specific needs of each neonate.

By automating the analysis of complex medical data and providing treatment recommendations, the proposed tool streamline clinical workflows. Neonatologist may focus more on direct neonate care and less on data interpretation and analysis. This efficiency improvement may enhance the overall productivity of healthcare teams.

Further, the disclosed tool's ability to interact with external devices and access various sources of patient data, such as electronic health records and monitoring equipment, facilitates seamless data integration. This integration simplifies the process of gathering essential information for making treatment decisions, reducing the administrative burden on clinicians.

The tool may collect data on treatment outcomes and receive feedback from clinicians. This valuable information may be used to refine and enhance the ML models over time. Continuous improvement ensures that the tool performance and accuracy evolve to meet the changing needs of healthcare providers and their patients. Assistance from the disclosed tool may help to reduce the occurrence of errors or oversights in treatment decisions. In critical neonatal cases, where the margin for error is narrow, the tool may provide an additional layer of support to enhance patient safety.

Moreover, in situations where neonatal care specialists are not physically present, the tool may offer the capability for remote consultation. Healthcare providers in various locations may access expert guidance and recommendations, ensuring that neonates receive the best possible care, regardless of geographical constraints.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A method for assisting clinicians in making real time decisions for resuscitation modalities in neonatal shock syndromes, the method comprising:
receiving, by a clinical decision support tool, medical data corresponding to a neonate diagnosed with shock;
extracting, by the clinical decision support tool, one or more features from the medical data, wherein the one or more features are indicative of perfusion status, possible etiology, type, and severity of shock, in the neonate;
selecting, by the clinical decision support tool, at least one machine learning model (ML) from a plurality of ML models based on the one or more features;
predicting, by the clinical decision support tool and via at least one ML model, an optimal treatment modality for the neonate, wherein the optimal treatment modality is one of fluid boluses or pressor support; and
assisting, by the clinical decision support tool, a clinician to provide the optimal treatment modality to the neonate based on predicting, wherein assisting specifies a number and type of the fluid boluses for the neonate, or a type and dose of the pressor support for the neonate, and wherein the assisting is based on the etiology and severity of shock in the neonate.

2. The method of claim 1, further comprising:
training the at least one ML model, wherein the at least one ML model is trained on a dataset of the medical data and the one or more features extracted.

3. The method of claim 2, further comprising:
testing the at least one ML model on new data to evaluate accuracy and performance of the at least one ML model.

4. The method of claim 2, further comprising:
optimizing the at least one ML model by adjusting hyperparameters, extracting different features from the one or more features, or combining two or more ML models from the plurality of ML models, based on feedback received from the clinician; and
deploying at least one ML model optimized in clinical settings to assist the clinician in making informed treatment decisions.

5. The method of claim 1, wherein the medical data is received from one or more of electronic health records, bedside monitors, laboratory tests, imaging devices, or wearable sensors.

6. The method of claim 1, wherein the medical data comprises medical history of the neonate, vital signs of the neonate, laboratory results of the neonate, and other related clinical and demographic information of the neonate.

7. The method of claim 1, wherein the plurality of ML models comprises decision trees or random forests, support vector machines (SVMs), gradient boosting, recurrent neural networks (RNNs) or long short-term memory (LSTM), transformers, and Bayesian methods.

8. A clinical decision support tool for assisting clinicians in making real time decisions for resuscitation modalities in neonatal shock syndromes, the clinical decision support tool comprising:
a processor (104); and
a memory (202) communicatively coupled to the processor, wherein the memory (202) stores processor instructions, which when executed by the processor (104), cause the processor (104) to:
receive medical data corresponding to a neonate diagnosed with shock;
extract one or more features from the medical data, wherein the one or more features are indicative of perfusion status, possible etiology, type, and severity of shock, in the neonate;
select at least one machine learning model (ML) from a plurality of ML models based on the one or more features;
predict, via at least one ML model, an optimal treatment modality for the neonate, wherein the optimal treatment modality is one of fluid boluses or pressor support; and
assist a clinician to provide the optimal treatment modality to the neonate based on predicting, wherein assisting specifies a number and type of the fluid boluses for the neonate, or a type and dose of the pressor support for the neonate, and wherein the assisting is based on the etiology and severity of shock in the neonate.

9. The clinical decision support tool of claim 8, wherein the processor instructions, on execution, further cause the processor to:
train the at least one ML model, wherein the at least one ML model is trained on a dataset of the medical data and the one or more features extracted.

10. The clinical decision support tool of claim 9, wherein the processor instructions, on execution, further cause the processor to:
test the at least one ML model on new data to evaluate accuracy and performance of the at least one ML model.

11. The clinical decision support tool of claim 9, wherein the processor instructions, on execution, further cause the processor to:
optimize the at least one ML model by adjusting hyperparameters, extracting different features from the one or more features, or combining two or more ML models from the plurality of ML models, based on feedback received from the clinician; and
deploy at least one ML model optimized in clinical settings to assist the clinician in making informed treatment decisions.

12. The clinical decision support tool of claim 8, wherein the medical data is received from one or more of electronic health records, bedside monitors, laboratory tests, imaging devices, or wearable sensors.

13. The clinical decision support tool of claim 8, wherein the medical data comprises medical history of the neonate, vital signs of the neonate, laboratory results of the neonate, and other related clinical and demographic information of the neonate.

14. The clinical decision support tool of claim 8, wherein the plurality of ML models comprises decision trees or random forests, support vector machines (SVMs), gradient boosting, recurrent neural networks (RNNs) or long short-term memory (LSTM), transformers, and Bayesian methods.

15. A non-transitory computer-readable medium storing computer-executable instructions for assisting clinicians in making real time decisions for resuscitation modalities in neonatal shock syndromes, the computer-executable instructions configured for:
receiving medical data corresponding to a neonate diagnosed with shock;
extracting one or more features from the medical data, wherein the one or more features are indicative of perfusion status, possible etiology, type, and severity of shock, in the neonate;
selecting at least one machine learning model (ML) from a plurality of ML models based on the one or more features;
predicting, via at least one ML model, an optimal treatment modality for the neonate, wherein the optimal treatment modality is one of fluid boluses or pressor support; and
assisting a clinician to provide the optimal treatment modality to the neonate based on predicting, wherein assisting specifies a number and type of the fluid boluses for the neonate, or a type and dose of the pressor support for the neonate, and wherein the assisting is based on the etiology and severity of shock in the neonate.
